(19) United States
(12) Patent Application Publication
(10) Pub. No.: US 2021/0402017 A1
(43) Pub. Date: Dec. 30, 2021

(54) METHOD FOR INACTIVATING A VIRUS ON A SURFACE

(71) Applicant: MERCK PATENT GMBH, DARMSTADT (DE)

(72) Inventors: Howard EPSTEIN, Leicester, NC (US); Qinyun PENG, Columbus, NJ (US); Bettina MAGSAAM, Michelstadt (DE); Heike SCHUCHMANN, Rossdorf (DE); Paul NEWCOMB, East Norriton, PA (US); Jutta Zur LAGE, Darmstadt (DE)

(73) Assignee: MERCK PATENT GMBH, DARMSTADT (DE)

(21) Appl. No.: 17/357,129

(22) Filed: Jun. 24, 2021

Related U.S. Application Data
(60) Provisional application No. 63/044,026, filed on Jun. 25, 2020.

(51) Int. Cl. A61L 2/00

(52) U.S. Cl. CPC A61L 2/0088

(57) ABSTRACT

The present invention is related to a method for inactivating a virus on a surface, especially in relation to a SARS-CoV-2 virus, to a composition capable of inactivating a virus on a surface as well as to the use of such a composition for the inactivation of a virus on a surface.

The present invention is related to a method for inactivating a virus on a surface, especially in relation to a SARS-CoV-2 virus, to a composition capable of inactivating a virus on a surface as well as to the use of such a composition for the inactivation of a virus on a surface.

Currently there is global concern related to the pandemic of a novel corona virus named SARS-CoV-2 virus. To fight the spread of infection, health authorities like WHO, CDC and FDA advise using personal protection in private and professional use. Such personal protection include face masks, hand sanitizers, disinfection agents, protective clothing, gloves, face shields and the like.

OTC products for private use often contain at least 70 vol % of alcohol or hydrogen peroxide, since these substances have been used for decades to fight viruses and promise to be effective against SARS-CoV-2 as well.

Although products containing such a large amount of alcohols or hydrogen peroxide are in principle suitable for application to human skin or pet's skin, it is not desirable to have frequent contact therewith because both substances may damage the skin barrier, leading to dehydrating effects and skin irritation.

With respect to cosmetic and hygienic products, consumers nowadays seek natural, gentle, safe and effective ingredients for their personal skin care. Under the current clean cosmetic trend, consumers also prefer products with multi-functional properties with least amount of ingredients in a product. Associated with this growing trend is the desire of the consumers to have minimal and preferable no preservatives in their skin care products. Therefore, there is a need for gentle viricidal materials effective against viruses, especially the SARS-CoV-2 virus, providing additional positive effects to the skin or any other surface treated therewith.

Thus, the object of the present invention is to provide a method for inactivating a virus on a surface, especially inactivating the SARS-CoV-2 virus, wherein the surface is not restricted to human skin and hair, and

wherein a gentle product with low tox profile is capable of inactivating a virus, especially the SARS-CoV-2 virus and, in addition, may provide additional positive effects to the surface treated therewith.

In addition, it is an object of the present invention to find a composition which is capable of inactivating enveloped as well as non-enveloped viruses including the SARS-CoV-2 virus on a variety of different surfaces, may be provided in different application forms and is capable of providing at least one further positive effect to the surface treated therewith.

Still furthermore, it is an object of the present invention to suggest an appropriate use of a composition capable of providing the effects mentioned above.

The object of the present invention is achieved by a method for inactivating a virus on a surface by applying a composition onto that surface, said composition comprising an amount of ethyl 3-(N-butylacetamino) propionate effective to inactivate a virus, thereby distributing said composition on that surface and maintaining it for a period of at least 30 seconds.

In addition, the object of the present invention is achieved by a composition capable of inactivating a virus on a surface, said composition comprising ethyl 3-(N-butylacetamino) propionate in an amount of from 0.1 to 100% by weight, based on the total weight of the composition.

Furthermore, the object of the present invention is also achieved by the use of a composition as described above for inactivating a virus on a surface, the surface being human skin, human hair, pet's skin or pet's fur, or being of woven or non-woven fabric, or is a textile product of natural or synthetic fibers or mixtures thereof, or is a surface of wood, metal, rubber, concrete, ceramics, porcelain, glass, leather, paper, cardboard, polymers or any combination thereof.

Ethyl 3-(N-butylacetamino) propionate is a substance known for years and was originally developed for insect repellent use. The substance is based on the naturally occurring beta-amino acid found in people and has a very low tox profile. It is used in insect repellent products for fighting e.g. mosquitoes including the mosquito transmitting the Zika virus, ticks, ants, cockroaches and other biting or crawling insects. In addition, it might successfully be used in agents against head lice or dust mites. Applications in sunscreen products are also available.

Ethyl 3-(N-butylacetamino) propionate is registered under the IUPAC-name 3-(N-n-Butyl-N-acetyl)aminopropionic acid ethyl ester and has the CAS number 52304-36-6. The product is available from EMD Performance Materials Corp., USA, or Merck KGaA, Germany, under the registered trademark IR3535®.

Compositions comprising ethyl 3-(N-butylacetamino)-propionate are, in addition to their insect repellent activity, effective against bacteria, yeast and molds. The respective formulations are gentle in their application and might be used on babies' skin and atopic skin as well without imparting skin irritation, inflammation or dehydration.

Similar to human skin or hair, formulations containing ethyl 3-(N-butylacetamino) propionate may be applied to pet's skin and pet's fur, in particular of dogs, cats or horses which are animals being in close contact to humans.

Here, these formulations act the same way as on human skin or human hair.

Insect repellent formulations containing ethyl 3-(N-butylacetamino) propionate exhibit a long-lasting repellence over a number of hours and may remain on their application field for hours. They usually exhibit high chemical and thermal stability.

In addition to biological application media, it has also turned out that even products made of fabrics may be treated with insect repellent formulations containing ethyl 3-(N-butylacetamino) propionate, thereby maintaining the insect repellent characteristics.

Since formulations containing ethyl 3-(N-butylacetamino) propionate exhibit numerous benefits in their application and effectiveness, there was the question arising whether additional positive effects might be achieved in respect to further positive effects not revealed so far.

Surprisingly, it has recently turned out that formulations containing ethyl 3-(N-butylacetamino) propionate do also exhibit viricidal properties.

Thus, the present inventors herewith provide a method for inactivating a virus on a surface, wherein a composition is applied onto that surface, and wherein said composition comprises an amount of ethyl 3-(N-butylacetamino) propionate effective to inactivate a virus, whereby said composition is distributed on that surface and is maintained there for a period of at least 30 seconds.

Ethyl 3-(N-butylacetamino) propionate is abbreviated to read EBAAP in the following.

Compositions comprising EBAAP exhibit viricidal properties against enveloped and non-enveloped viruses such as the non-enveloped model viruses human adenovirus Type 5 (Adenoid strain 75, ATCC VR-5) and human poliovirus Type 1 (Chat strain, ATCC VR-1562), as well as against the enveloped SARS-CoV-2 virus.

When viricidal property is established, it has been found appropriate to establish the effectiveness by means of selected critical model viruses in order to conclude to a general viricidal property based on of such results. This approach is supported by the Directions of the German Federal Public Health Administration and the Deutsche Vereinigung zur Bekämpfung der Viruskrankheiten of 1982. These directions have been updated in 2004 by a special working group of the Robert Koch Institute (RKI) and other authorities.

According to recommendations of the RKI, preparations claiming viricidal effectiveness are tested in the Federal Republic of Germany against, inter alia, the model viruses adenovirus type 5 (strain adenoid 75) and poliovirus strain type 1. A preparation is considered effective against the respective test virus if a reduction of the virus titer of at least 4 logarithmic steps is achieved in a quantitative suspension experiment.

According to the present method, a composition comprising an amount of EBAAP effective to inactivate a virus is applied to a receiving surface, distributed thereon and maintained thereon for at least 30 seconds.

The application of the composition to the surface depends on the form of the composition and the kind of the receiving surface. The composition may be provided in the form of an oily liquid, an aqueous solution, an aqueous-alcoholic solution, an aerosol, a lotion, an emulsion, a cream, a gel, a shampoo, a stick, a bar, a wipe or a towelette. Such application forms are especially useful for use on human skin, human hair, pet's skin or pet's fur.

Regarding use for human beings or animals, the composition may be used for professional clinic applications as well as for private use in OTC products.

The composition is usually sprayed onto the surface and distributed either by spraying a liquid formulation onto the full area of the surface to be treated or by distributing the liquid formulation by hand or by other means, e.g. a tissue, a paper, a brush, etc. Other application methods are rubbing the surface area to be treated with the composition, immersing the surface to be treated, or any other useful method. In case the composition is a soap bar, the surface to be treated may be simply washed with water and the bar.

The method according to the present invention does also comprise the treatment of inanimate surfaces such as woven or non-woven fabrics, textile products of natural or synthetic fabrics or of mixtures thereof, or even of surfaces of wood, metal, rubber, concrete, ceramics, porcelain, glass, leather, paper, cardboard, polymers or any combination thereof.

In these cases, special clothing treatment compositions or liquid compositions in general may be advantageously be used, since these allow easy application and distribution of the composition on the surface to be treated. The pure oily liquid compound, aqueous solutions, aqueous-alcoholic solutions or aerosols are especially useful for inanimate surfaces. Usually, the respective surface is sprayed with the respective composition or immersed therein. Of course, other application forms such as wiping by pieces of fabrics or paper treated with EBAAP are also useful.

Since the composition comprising EBAAP as active compound is not only gentle to animate surfaces such as human skin but does also not react harshly against inanimate surfaces such as wood, steel, glass, leather etc., it allows being applied onto large surfaces such as walls, ceilings, working benches, floors and floor coverings, to name only a few, to defend viruses. In addition, the viricidal treatment of personal protection means such as masks, protection shields, gloves, door handles, as well as the treatment of medical devices such as surgical instruments, hospital equipment or even household articles is also possible.

After being applied and distributed on the surface to be treated, the composition comprising EBAAP shall be maintained on the surface for a period of at least 30 seconds to establish the viricidal activity, i.e. to kill the respective virus.

Usually, the time period for providing a viricidal activity on the respective surface is from 30 seconds to 5 minutes, especially from 30 seconds to 2 minutes.

Nevertheless, since the composition comprising EBAAP does not harmfully react on the surface to be treated, it may remain there up to the next washing, cleaning or viricidal treating procedure.

EBAAP itself does not exhibit any aggressive or harsh reaction against animate or inanimate surfaces, such as human skin, metal or glass. Nonetheless, the reason why the composition used according to the present invention does not react aggressive or harsh against any surface is that it contains only a small amount of any alcohol or of hydrogen peroxide, if at all. Due to the viricidal and antibacterial characteristics of EBAAP, the amount of alcohols or hydrogen peroxide may be reduced to a great extent. Therefore, amounts of less than 50% by weight of any alcoholic compound or of hydrogen peroxide, related to the weight of the composition in total, are possible.

On the other side, the viricidal activity of any alcoholic composition or hydrogen peroxide, if present in the composition according to the present invention, is boosted to such an extent by the content of EBAAP, that amounts of alcohols, which usually are present to at least 60 or 70% by weight in commercially available viricidal compositions, may be avoided. Therefore, the amount of alcohols used in the present composition may be reduced to at most 40% by weight, based on the weight of the composition. Preferably, the amount of any alcohol is reduced to 0 to 35% by weight, preferably to 0 to 20% by weight, based on the weight of the composition in total. In an especially preferred embodiment, there is no alcohol or hydrogen peroxide contained in the composition.

The content of EBAAP in the composition used in the method according to the present invention is 0.1 to 100% by weight, based on the weight of the composition in total. Preferably, the composition comprises 0.5 to 50% by weight of EBAAP.

It goes without saying, that the content of the active compound EBAAP in the respective composition depends on the kind of the composition.

In case the composition is in a liquid state, the content of EBAAP is usually between 2 and 100% by weight, based on the weight of the composition in total. For most application forms, contents in the range of from 2 to 50% by weight, especially in the range of from 10 to 30% by weight, are sufficient and effective.

In case the composition is in a solid state such as in the form of a cream, a stick or a bar, the content of the active compound EBAAP is in most cases also in the range of from 5 to 30% by weight, especially in the range of from 10 to 25% by weight.

In case the composition is in the form of a wipe or a towelette, treated with EBAAP, the content of the active compound may be much less, for example may be in the range of from 0.1 to 10% by weight, preferably in the range of from 0.5 to 5% by weight, based on the weight of the composition. Here, a big relative amount of the composition is occupied by the paper, tissue or fabric treated with EBAAP, either in pure liquid form or in form of a liquid composition containing EBAAP. The fabric is preferably composed of cotton and/or viscose fibers and may comprise synthetic fibers as well. Depending on the application form, the composition containing EBAAP used in the present method may also comprise other ingredients which are typically used in cosmetic and pharmaceutical compositions and are considered physiologically acceptable.

A liquid composition, emulsion or suspension used in the present process may comprise water, which is usually included in demineralized form. The content thereof varies in a broad range and may be selected depending on the application form and the surface to be treated.

The composition to be used in the present method, especially in case of an aqueous-alcoholic solution, may also comprise an alcohol. As mentioned above, the content of alcohol shall be limited to at most 40% by weight, based on the weight of the composition in total. Values in the range of 0 to 35% by weight, especially 0 to 20% by weight, are preferred. In a particularly preferred embodiment, the composition does not comprise any alcohol.

Suitable alcohols are for example ethanol, propane-1-01, propane-2-ol and mixtures of any of these.

Auxiliaries which are usually used in liquid phases of cosmetic or pharmaceutical compositions are also diols or polyols of low carbon number as well as ethers thereof, preferably propylene glycol, glycerol, ethylene glycol, ethylene glycol monoethyl ether, ethylene glycol monobutyl ether, propylene glycol monomethyl ether, propylene glycol monoethyl ether, propylene glycol monobutyl ether, diethylene glycol monomethyl ether, diethylene glycol monoethyl ether, etc. They may be used in the composition used according to the present method in amounts in the range of from 1 to 15% by weight, based on the weight of the composition in total.

Optionally, the composition used in the present process may also comprise further additives such as a fragrance, a dyestuff, a fat, a wax, an oil, a binder, a surfactant, a thickener, a smoothing agent, a moisturizer, a gelling agent, a skin care component, a solubilizer, a stabilizer, an emulsifier, an anti-inflammatory agent, a vitamin, a denaturating agent, a refatting agent, a UV photoprotective filter, an antioxidant, an organic pigment, an inorganic pigment, as well as a further bactericidal or viricidal compound.

These additives may be used either alone or as a mixture of two or more thereof and may be combined with each other depending on the application form and the surface to be treated.

Since these components and the respective materials are well known in the art being usually comprised in cosmetic or pharmaceutical compositions, only a few will be listed in the following for example.

Thickeners may be silicon dioxide, aluminum silicates polysaccharides or derivatives thereof, for example hyaluronic acid, xanthan gum, hydroxy methylcellulose and, especially, various polyacrylates. Polyurethane compounds, especially water soluble or water dispersible anionic polyurethanes may also be used as thickeners.

Preferred thickeners are natural occurring materials such as agar, guar gum, alginates, gum arabic, locust bean gum, linseed gums, dextrans and various starch fractions and derivatives. Polyvinyl alcohol as well as partially saponified polyvinyl acetates and their derivatives may also be used.

In addition, organic gelling agents of natural or synthetic origin, such as polyacrylamide or polyvinylpyrrolidone and their derivatives may be used-

A great variety of oils, fats and waxes may be used as well, which are usually used for skin care reasons. These compounds include bisabolol, tocopheryl acetate, dodecanol, tetradecanol, hexadecanol, octadecanol, lanolin alcohol, cetearyl alcohol, cyclomethicone, dimethicone, isopropyl myristate, isopropyl palmitate, cetearylethyl hexanoate, octyl stearate, octyl octanoate, ethylhexanoic acid ethyl ester, jojoba oil, sea buckthorn oil, wool wax, paraffin oil, vaseline, cholesterol and various glycerides.

Moisturizers and anti-inflammatories comprise for example glycerol, propane-1,2-diol, butane-1,3-diol, sorbitol, dexpanthenol and allantoin.

Refatting agents which prevent the surface which is human or animal skin from drying out may be selected for example from glycerol trioleate, glycerol dioleate, glycerol monooleate, glyceryl caprylate, glyceryl caprate or fatty alcohols. Isopropyl myristate and cetearyl octanoate are useful as well.

Ethyl 3-(N-butylacetamino) propionate (EBAAP) exhibits a bactericidal activity against Escherichia coli, Klebsiella pneumoniae, Staphylococcus aureus, Bacillus subtilis and Acinetobacter baumannii and is also effective against yeast (Candida albicans) and fungi (Aspergillus niger, Bortrytis cinerea and Penicillium vinaceum). Therefore, by applying a composition comprising EBAAP in an amount effective to deactivate a virus onto a surface, distributing said composition and maintaining it for a period of at least 30 seconds, the surface is also provided with bactericidal properties.

Since it is known that EBAAP containing compositions are effective against various insects by repelling them over a period of several hours, an insect repelling activity is also provided to the surface treated with the composition used in the present method.

The present invention is also directed to a composition capable of inactivating a virus on a surface, said composition comprising ethyl 3-(n-butylacetamino) propionate in an amount of from 0.1 to 100% by weight, based on the weight of the composition in total.

Preferably, the content of the active compound EBAAP in the present composition is in the range of from 0.5 to 50% by weight, based on the weight of the composition in total.

For details regarding to the particularly preferred contents of EBAAP in liquid or solid compositions, reference is made to the respective passages disclosed above.

This composition is effective against enveloped and non-enveloped viruses on animate and inanimate surfaces.

This composition is especially effective against the SARS-CoV-2 virus on a surface.

The different surfaces onto which the present composition may be applied have already been disclosed earlier. Insofar, reference is made to this passage of the present description.

As already disclosed before, the composition according to the present invention may comprise other ingredients which are typically used in cosmetic and pharmaceutical compositions and are considered physiologically acceptable.

Optionally, the composition used in the present process may also comprise further additives such as a fragrance, a dyestuff, a fat, a wax, an oil, a binder, a surfactant, a thickener, a smoothing agent, a moisturizer, a gelling agent, a skin care component, a solubilizer, a stabilizer, an emulsifier, an anti-inflammatory agent, a vitamin, a denaturating agent, a refatting agent, a UV photoprotective filter, an antioxidant, an organic pigment, an inorganic pigment, as well as a further bactericidal or viricidal compound.

These additives may be used either alone or as a mixture of two or more thereof and may be combined with each other depending on the application form and the surface to be treated.

In order to avoid skin irritating, inflammating and other annoying effects on the surfaces treated, the content of alcohol in the composition according to the present invention shall be restricted to at most 40% by weight, based on the weight of the composition in total. Values in the range of 0 to 35% by weight, especially 0 to 20% by weight, are preferred. In a particularly preferred embodiment, there is no alcohol comprised in the composition. Suitable alcohols, if present, are for example ethanol, propane-1-ol, propane-2-ol and mixtures of any of these.

Hydrogen peroxide or any other oxygen releasing compound is preferably not comprised in the composition according to the present invention.

The composition according to the present invention is provided in the form of an oily liquid, an aqueous solution, an aqueous-alcoholic solution, an aerosol, a lotion, an emulsion, a cream, a gel, a shampoo, a stick, a bar, a wipe or a towelette.

A special form of a liquid composition is a clothing treatment composition.

These application forms of cosmetic or pharmaceutical compositions are known per se as well as their main ingredients, so that the skilled person can produce like products in standard mixing procedures and with standard ingredients besides the content of EBAAP and the necessarily low content of alcohols in the respective composition.

The present invention is also directed to the use of a composition as described above for inactivating a virus or a surface, the surface being human skin, human hair, pet's skin or pet's fur, or being of woven or non-woven fabric, or is a textile product of natural or synthetic fibers or mixtures thereof, or is a surface of wood, metal, rubber, concrete, ceramics, porcelain, glass, leather, paper, cardboard, polymers or any combination thereof.

While inactivating a virus on said surface, the surface treated with the composition according to the present invention is also provided with bactericidal and insect repellent properties, due to the intrinsic multi-functionality of the active substance ethyl 3-(N-butylacetamino) propionate.

The present invention is further illustrated by the following examples without being limited thereto.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding and U.S. Provisional Application Ser. No. 63/044,026, filed Jun. 25, 2021, are incorporated by reference herein.

### EXAMPLES

### Example 1

Hand Sanitizer Gel

| | | | |
|---|---|---|---|
| Ingredients | No. | INCI | % |

Phase A

| Water, demineralized | | AQUA (WATER) | add. 100 |
|---|---|---|---|
| Carbopol Ultrez 21 Polymer | (2) | ACRYLIC | 0.75 |
| | | POLYMER | |
| Triethanolamine | | TRIETHANOLAMINE | 0.70 |

Phase B

| | | | |
|---|---|---|---|
| 1,3 BG | | BUTYLENE | 6.00 |
| | | GLYCOL | |
| RonaCare ® | (1) | ALLANTOIN | 0.10 |
| Allantoin | | | |
| RonaCare ® | (1) | CETRIMONIUM | 0.10 |
| Cetrimonium | | BROMIDE | |
| Bromide | | | |
| IR3535 ® | (1) | ETHYL | 20.00 |
| | | BUTYLACETYLAMINOPROPIONATE | |
| Ethanol 96% | (1) | ALCOHOL | 19.96 |

Procedure:
1. Pre-wet U-21 for 10 min then stir to slurry.
2. Add TEA into phase A then make into gel. Slow down the stirring to avoid much air bubble.
3. Add all ingredients of phase B into Alcohol and mix until clear solution is obtained.
4. Add phase B into A with fast speed and also speed up the stir speed to make a semi-transparent gel.

pH: 6.8

Suppliers: (1) Merck KGaA, Darmstadt Germany/EMD Performance Materials Corp. USA
(2) Lubrizol

### Example 2

Liquid Hand Soap

| | | | |
|---|---|---|---|
| Ingredients | No. | INCI | % |

Phase A

| Water, demineralized | | AQUA (WATER) | add. 100 |
|---|---|---|---|
| RonaCare ® Biotin Plus | (1) | UREA (AND) | 0.50 |
| | | DISODIUM PHOSPHATE | |
| | | (AND) BIOTIN (AND) | |
| | | CITRIC ACID | |
| RonaCare ® CPC | (1) | CETYLPYRIDINIUM | 0.20 |
| | | CHLORIDE | |
| Triplex ® III | (1) | DISODIUM EDTA | 0.05 |
| Glycerol, anhydrous | (1) | GLYCERIN | 3.00 |
| Calamide ® MC | (2) | COCAMIDE MEA | 0.50 |

Phase B

| | | | |
|---|---|---|---|
| Carbopol ® Aqua | (3) | ACRYLATES POLYMER | 8.00 |
| SF-1 Polymer | | | |
| ColaDEt EQ-18 | (4) | DECYL GLYCOSIDE (AND) | 30.00 |
| | | COCAMIDOPROPYL HYDROXY- | |
| | | SULTAINE (AND) | |
| | | COCAIMDOPROPYL | |
| | | BETAINE (AND) COCAMIDE | |
| | | MIPA (AND) DISODIUM | |
| | | LAURETH-12 SULFOSUCCINATE | |
| IR3535 ® | (1) | ETHYL | 10.00 |
| | | BUTYLACETYLAMINOPROPIONATE | |
| Xirona ® | (1) | IRON OXIDES (AND) | 0.08 |
| Le Rouge | | SILICA | |
| Timiron ® | (1) | BISMUTH OXYCHLORIDE | 0.08 |
| Liquid | | (AND) ETHYL | |
| Silver | | HYDROXYSTEARATE | |
| Fragrance | | FRAGANCE | q.s. |

Phase C

| | | | |
|---|---|---|---|
| Lincoserve ™ WpH-LO | (5) | PENTYLENE GLYCOL (AND) | 1.50 |
| | | PROPANEDIOL (AND) | |
| | | CAPRYLYL GLYCOL (AND) | |
| | | ETHYLHEXYL GLYCERIN | |
| RonaCare ® Sodium | (1) | WATER (AND) SODIUM | q.s. |
| Hydroxide about 32% | | Solution HYDROXIDE | |

Procedure:

In a suitable beaker, add DI water. Then, add the remaining ingredients of phase A, one ingredient at the time with moderate mixing while heating to 75-80° C. until Calamide® MC dissolved. Then, cool to 40° C., add phase B ingredients, one at a time. Continue mixing and cooling. Do not incorporate air into the batch. Add phase C, then adjust to pH 6-0-7.0 with the sodium hydroxide solution.

Pearlescent gel, pH: 6.3

Suppliers: (1) Merck KGaA, Darmstadt Germany/EMD Performance Materials Corp. USA
(2) Pilot Chemical Company
(3) Lubrizol
(4) Colonial Chemical, Inc.
(5) Lincoln MFG-USA

### Example 3

Aqueous-Alcoholic Solution for Wipe Production

| | | | |
|---|---|---|---|
| Ingredients | No. | INCI | % |

Phase A

| | | | |
|---|---|---|---|
| IR3535 | (1) | ETHYL | 20.00 |
| | | BUTYLACETYLAMINOPROPIONATE | |
| Polyethylene | (1) | PEG-8 | 5.00 |
| Glycol 400 | | | |
| Arlamol PS15E | (2) | PPG-15 STEARYL ETHER | 3.00 |
| Fragrance | | PARFUM | q.s. |

Phase B

| | | | |
|---|---|---|---|
| Ethanol 96% | (1) | ALCOHOL | 35.00 |
| MP Diol Glycol | (3) | METHYL PROPANEDIOL | 4.00 |
| Twen 20 | (1) | POLYSORBATE 20 | 1.50 |
| Water, demineralized | | AQUA | ad 100 |

Procedure:

Blend phase A and B separately. Incorporate phase B slowly into phase A and stir until a homogeneous and clear mixture is obtained.

Clear solution, pH: 6.5

Suppliers: (1) Merck KGaA, Darmstadt Germany/EMD Performance Materials Corp. USA
(2) Croda
(3) Lyondell Chemical, Rotterdam

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A method for inactivating a virus on a surface by applying a composition onto that surface, said composition comprising an amount of ethyl 3-(N-butylacetamino) propionate effective to inactivate a virus, thereby distributing said composition on that surface and maintaining it for a period of at least 30 seconds.

2. A method according to claim 1, wherein the virus is an enveloped virus, a non-enveloped virus, or both.

3. A method according to claim 1, wherein the virus is SARS-CoV-2 virus.

4. A method according to claim 1, wherein the surface is human skin, human hair, pet's skin or pet's fur.

5. A method according to claim 1, wherein the surface is of woven or non-woven fabric, or is a textile product of natural or synthetic fibers or mixtures thereof.

6. A method according to claim 1, wherein the surface is of wood, metal, rubber, concrete, ceramics, porcelain, glass, leather, paper, cardboard, polymers or any combination thereof.

7. A method according to claim 1, wherein the composition comprises ethyl 3-(N-butylacetamino) propionate in an amount of from 0.1 to 100% by weight, based on the total weight of the composition.

8. A method according to claim 7, wherein the composition comprises ethyl 3-(N-butylacetamino) propionate in an amount of from 0.5 to 50% by weight, based on the total weight of the composition.

9. A method according to claim 1, wherein the composition is in the form of an oily liquid, an aqueous solution, an aqueous-alcoholic solution, an aerosol, a lotion, an emulsion, a cream, a gel, a shampoo, a stick, a bar, a wipe or a towelette.

10. A method according to claim 1, wherein the composition optionally comprises a liquid medium, a fragrance, a dyestuff, a fat, a wax, an oil, a binder, a surfactant, a thickener, a smoothing agent, a moisturizer, a gelling agent, a skin care component, a solubilizer, a stabilizer, an emulsifier, an anti-inflammatory agent, a vitamin, a denaturating agent, a refatting agent, a UV photoprotective filter, an antioxidant, an organic pigment, an inorganic pigment, as well as a further bactericidal or viricidal compound.

11. A method according to claim 1, wherein said surface is additionally provided with bactericidal properties.

12. A composition capable of inactivating a virus on a surface, said composition comprising ethyl 3-(N-butylacetamino) propionate in an amount of from 0.1 to 100% by weight, based on the total weight of the composition.

13. A composition according to claim 12, wherein the composition is capable of inactivating an enveloped virus, a non-enveloped virus, or both of these.

14. A composition according to claim 12, wherein the composition is capable of inactivating the SARS-CoV-2-virus on a surface.

15. A composition according to claim 12, comprising ethyl 3-(N-butylacetamino) propionate in an amount of from 0.5 to 50% by weight, based on the total weight of the composition.

16. A composition according to claim 12, wherein the composition optionally comprises a liquid medium, a fragrance, a dyestuff, a fat, a wax, an oil, a binder, a surfactant, a thickener, a smoothing agent, a moisturizer, a gelling agent, a skin care component, a solubilizer, a stabilizer, an emulsifier, an anti-inflammatory agent, a vitamin, a denaturating agent, a refatting agent, a UV photoprotective filter, an antioxidant, an organic pigment, an inorganic pigment, as well as a further bactericidal or viricidal compound.

17. A composition according to claim 12, wherein the composition is in the form of an oily liquid, an aqueous solution, an aqueous-alcoholic solution, an aerosol, a lotion, an emulsion, a cream, a gel, a shampoo, a stick, a bar, a wipe or a towelette.

18. A composition according to claim 12, wherein the composition does contain at most 40% by weight of alcohol, based on the total weight of the composition.

19. A composition according to claim 12, wherein the composition does not contain any alcohol or hydrogen peroxide.

20. A method for inactivation of a virus on a surface comprising contacting the composition of claim 12 to the surface, the surface being human skin, human hair, pet's skin or pet's fur, or being of woven or non-woven fabric, or is a textile product of natural or synthetic fibers or mixtures thereof, or is a surface of wood, metal, rubber, concrete, ceramics, porcelain, glass, leather, paper, cardboard, polymers or any combination thereof.

21. The method according to claim 20, wherein the surface is also provided with bactericidal properties.
